(19) United States
(12) Patent Application Publication
(10) Pub. No.: US 2003/0013994 A1
(43) Pub. Date: Jan. 16, 2003

(54) Methods and systems for generating a prognosis

(76) Inventors: Gilles Rubinstenn, Paris (FR); Francis Pruche, Senlis (FR)

Correspondence Address: FINNEGAN, HENDERSON, FARABOW, GARRETT & DUNNER, L.L.P., 1300 I Street, N.W., Washington, DC, 20005-3315, US

(21) Appl. No.: 10/024,333

(22) Filed: Dec. 21, 2001

Related U.S. Application Data
(60) Provisional application No. 60/325,559, filed on Oct. 1, 2001.
(60) Provisional application No. 60/304,046, filed on Jul. 11, 2001.

(51) Int. Cl. A61B 5/00

(52) U.S. Cl. 600/587

(57) ABSTRACT

A method enables determination of a prognosis for an external body condition of a subject. The method may involve receipt of one or more representations of the subject's external body condition. A prognosis may be generated based on both the representation and database information relating to how use of at least one beauty product affects evolution of the external body condition. The prognosis may then be outputted to enable the subject to receive the prognosis.

### BACKGROUND OF THE INVENTION

1. Field of the Invention

The invention relates to methods and systems for enabling determination of a prognosis of an external body condition of a subject. In one example, the invention may be used to generate at least one prognosis that reflects predicted changes in an external body condition after use of at least one beauty product.

2. Description of Related Art

Consumers today are faced with numerous choices of beauty products. For example, each beauty product has its own affect, efficacy, method of application, and frequency of use, making selection of particular product difficult and confusing. Traditionally, consumers have relied on product advertising or advice provided by numerous other sources, such as beauty consultants or articles in personal care magazines, to appreciate the impact a particular product or combination of beauty products might have on a particular external body condition. In addition to sometimes containing somewhat confusing or conflicting advice, many of these means of informative exchange leave a consumer with a degree of uncertainty about whether a particular product will ultimately provide an adequate remedy for a particular external body condition. Accordingly, consumers sometimes hesitate to purchase products.

Even after a consumer purchases a product and uses it for at least a short period of time, there could still be some doubt about whether the product will be effective in caring for a particular condition. In an attempt to gauge effectiveness, some consumers monitor their condition periodically to track any perceptible changes caused by a particular beauty product. Many beauty products, however, do not cause perceptible results that a consumer might expect, especially when a product has not been used for a significant period of time. By not being able to perceive expected changes somewhat immediately, at least some consumers tend to be discouraged with a beauty product (or combination of beauty products) they might be using. Thus, consumers are not only faced with many confusing choices with respect to purchase of beauty products, but also they have difficulty appreciating how those products might ultimately improve one or more of their conditions.

### SUMMARY OF A FEW ASPECTS OF THE INVENTION

One aspect of the invention may involve a method for enabling determination of a prognosis for an external body condition of a subject. The method includes receiving at least one representation of the subject's external body condition. The method also includes maintaining in a database information on how use of at least one beauty product affects evolution of the external body condition. The method further includes generating at least one prognosis reflecting predicted changes in the external body condition after use of the beauty care product(s), wherein the generating is based on both the representation and information contained in the database. The prognosis is output to enable the subject to receive the prognosis.

According to another aspect of the invention, a system for enabling determination of a prognosis for an external body condition of a subject is provided. The system includes a memory for receiving at least one representation of the subject's external body condition. The system further includes a database for storing information on how use of at least one beauty product affects evolution of the external body condition. The system further includes a processor for modifying the representation, based on information contained in the database, to generate at least one prognosis reflecting predicted changes in the external body condition after use of the beauty product. The system further includes a driver for outputting the prognosis to enable the subject to receive the prognosis.

In accordance with another aspect of the invention, a system for enabling determination of a prognosis of an external body condition of a subject is provided. The system includes means for receiving at least one representation of the subject's external body condition. The system further includes means for maintaining in a database information of how use of at least one beauty product affects evolution of the external body condition. The system further includes means for generating at least one prognosis reflecting predicted changes in the external body condition after use of the beauty product, wherein the generating is based on both the representation and information contained in the database. The system further includes means for outputting the prognosis to enable the subject to receive the prognosis.

According to another aspect of the invention, a system for enabling determination of prognosis for an external body condition of a subject is provided. The system includes a memory for receiving at least one image representative of the subject's external body condition. The system further includes a secondary storage storing a mesh frame representative of at least one part of human anatomy and a database containing information on how use of at least one beauty product affects evolution of the external body condition. The system further includes a processor for rendering the image on the wire mesh frame and modifying the image, based on information contained in the database, to generate at least one prognosis image reflecting predicted changes in the external body condition after use of said at least beauty product. The system further includes a driver for outputting the prognosis image to enable the subject to view the prognosis image.

According to yet another aspect of the invention, a computer readable medium containing instructions for a method for enabling determination of a prognosis for an external body condition is provided. The method includes receiving at least one representation of the subject's external body condition. The method also includes maintaining in a database information of how use of at least one beauty product affects evolution of the external body condition. The method further includes generating at least one prognosis reflecting predicted changes in the external body condition after use of the beauty product, wherein the generating is based on both the representation and information contained in the database. The prognosis is output to enable the subject to receive the prognosis.

As described hereafter, other aspects of the invention exist, for example, using computer graphics techniques to model various external body conditions and illustrating evolution over time of the various external body conditions. Thus, the summary of a few aspects of the invention is not to be interpreted as defining the invention.

### BRIEF DESCRIPTION OF THE DRAWINGS

The accompanying drawings, which are incorporated in and constitute a part of the specification exemplify the invention and together with the description, serve to explain the principles of the invention.

FIG. 1 is a diagram of a flow chart illustrating an exemplary method for enabling determination of a prognosis for an external body condition of a subject consistent with the present invention;

FIG. 2 is a schematic diagram illustrating a display showing output prognosis images consistent with the present invention;

FIG. 3 is a schematic diagram of a graph providing a prognosis consistent with the present invention;

FIG. 4 is a schematic diagram illustrating an example of a user interface enabling a subject to edit a representative image of an external body condition of the subject;

FIG. 5 is a schematic illustration of one example of a user interface for a user access device of FIG. 7;

FIG. 6 is an illustration of an exemplary environment in which the systems and methods consistent with the present invention may be implemented;

FIG. 7 is a schematic drawing of an exemplary user access device;

FIG. 8A is a schematic illustration of an exemplary embodiment of a computing module for the user access device of FIG. 7;

FIG. 8B is a schematic diagram illustrating exemplary modules that may be contained in a beauty care module shown in FIG. 8A;

FIG. 8C is a schematic illustration of an exemplary beauty products database;

FIG. 8D is a schematic illustration of a database containing images;

FIG. 9 is a schematic illustration of an exemplary embodiment of a computing module for a host site shown in FIG. 6;

FIG. 10A is an illustration of an exemplary facial skin mesh and corresponding muscle patches; and

FIG. 10B is an illustration of an exemplary model corresponding to a human face.

### DESCRIPTION OF EXEMPLARY EMBODIMENTS

Reference is now made in detail to exemplary embodiments of the invention, examples of which are illustrated in the accompanying drawings. Wherever possible, the same reference numbers are used throughout the drawings to refer to the same or like parts.

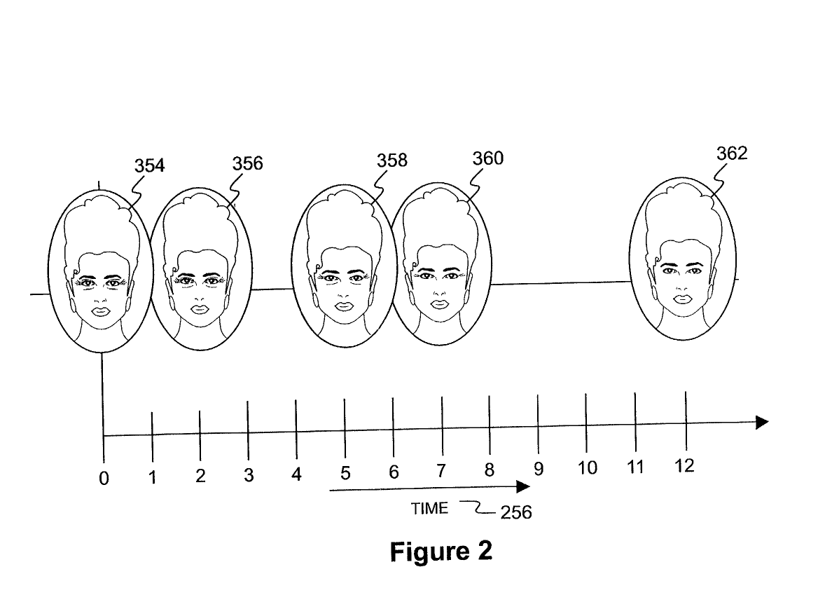

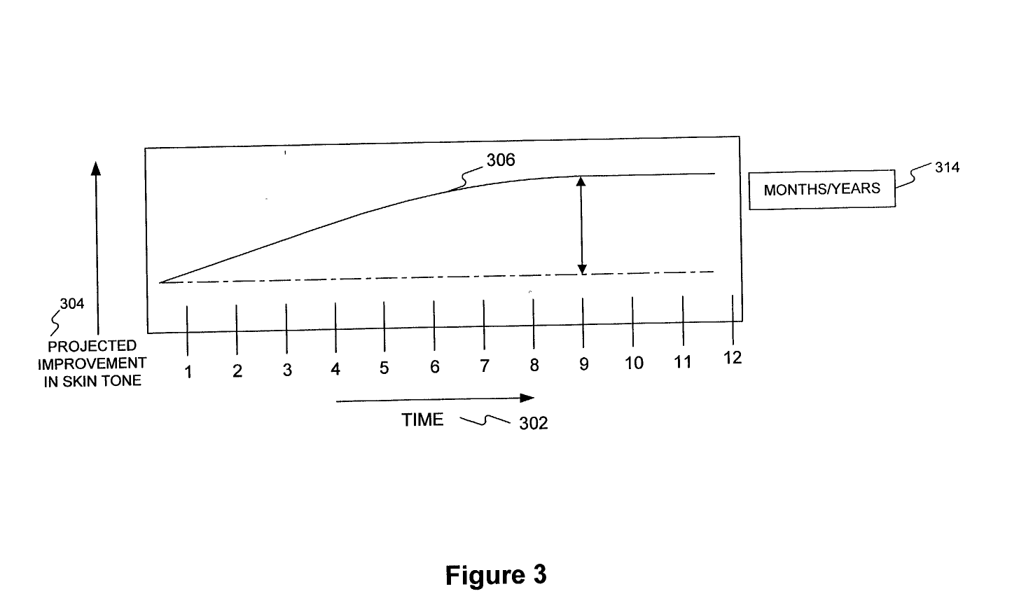

One aspect of the invention may involve a method and system for enabling a subject to determine the prognosis for an external body condition of the subject, assuming one or more beauty products will be used. As explained in more detail below, the method and system may involve generation and output of a prognosis reflecting predicted changes in the external body condition after use of the beauty product(s). As used herein, the term “prognosis” includes anything that provides a forecast of the probable evolution of an external body condition. The prognosis could be in many differing forms, such as a graph, image, chart, comparison, illustration, written information, audio information, and/or electronic information that can be constructed in any of the other forms, for example. The prognosis could be in the form of a graph, as shown in FIG. 3 explained below, or a modified image of an external body portion of the subject, as shown in FIG. 2 explained below, for example. The prognosis may be received (e.g., viewed) by the subject to give the subject an indication of the prognosis for the external body condition, and thereby assist the subject in deciding whether to initially use and/or purchase the beauty product and/or whether to continue existing use of the beauty product, for example.

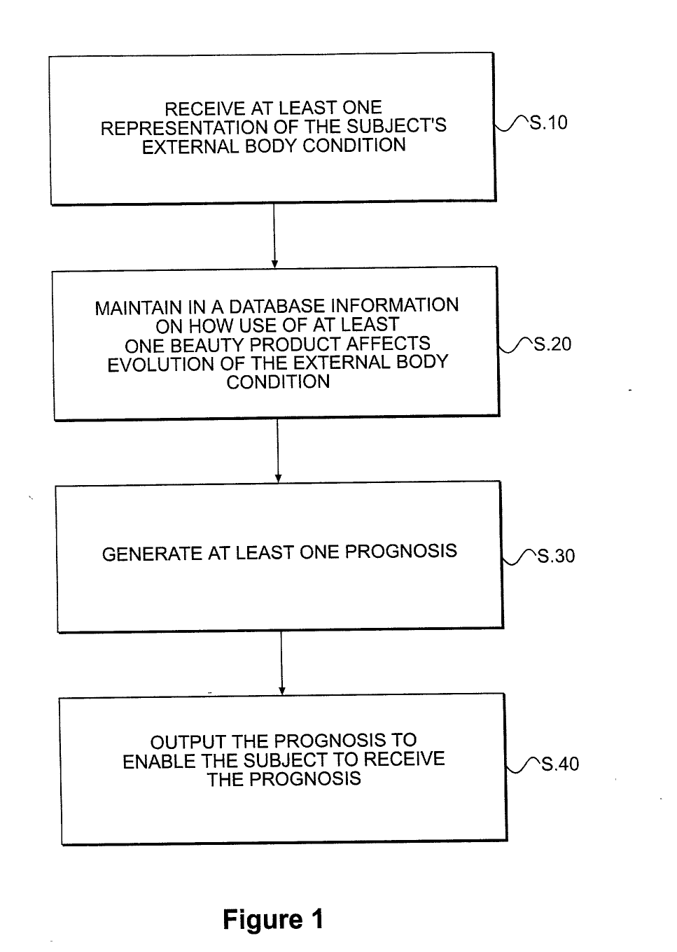

FIG. 1 is a flow chart showing one example of a method according to the invention. As explained in more detail below, the method may involve receiving at least one representation of the subject's external body condition (S. 10); maintaining in a database information of how use of at least one beauty product affects evolution of the external body condition (S. 20); generating at least one prognosis reflecting predicted changes in the external body condition after use of the beauty product (S. 30); and outputting the prognosis to enable the subject to receive the prognosis (S. 40).

As used herein, the term “external body condition” refers to one or more conditions of an external portion of a body. The condition could be a condition relating to any external body portion, such as the skin, hair, eyebrows, eyelashes, body hair, facial hair, fingernails, and/or toenails. Examples of conditions of the skin include, but are not limited to, skin texture, elasticity, dryness, cellulites, sweating, aging, wrinkles, melanoma, exfoliation, desquamation, homogeneity of color, micro-circulation, shininess, softness, smoothness, matitty, hydration, sebum production, cleanliness, irritation, redness, skin pore density and/or size, vasocolation, pigmentation, and freckles. Examples of conditions of the hair include, but are not limited to, thickness of hair strands, status of hair roots, split ends, oiliness, dryness, scales, keratin plugs, hair loss, and dandruff. Examples of conditions of fingernails and/or toenails include onychomycosis, split nails, delaminating, psoriasis, brilliancy, lines, and spots.

When the external body condition is a skin condition, the skin having the condition could be located on the subject's hand(s), foot/feet, arm(s), leg(s), and/or torso. When the external is a hair condition, the hair having the condition could be located on the subject's scalp, eyelashes, and/or eyebrows.

Additionally, as used herein, the term “subject” refers to the person or other life-form whose external body condition is taken into account in generating the prognosis. Optionally, one or more additional individuals could assist the subject or provide the subject with access to a user access device, for example. Such additional individuals include, but are not limited, cosmeticians, beauty consultants, sales agents at kiosks in stores or other places, or others, such as those who may assist the subject in evaluating the prognosis, for example.

As mentioned above, the method shown in FIG. 1 includes receiving one or more representations of the subject's external body condition (S.10). As used herein, the term “representation” includes anything providing an indication of the condition of an external body condition. Examples of representations includes, but are not limited to, test results, images, results of subjective evaluations, results of analytical and/or diagnostic techniques, etc. The following examples are described using a representation in the form of an image, but it should be understood that there are many other possible forms of representations. As used herein, the term “image” is intended to include either a visually perceptible representation or electronic image data that may be used to construct a visually perceptible represenation, for example. The image may be a likeness corresponding to an anatomical portion of the subject. For example, the image may be an image of the subject's entire face, or a portion of the subject's face. The image may be a detailed picture (e.g., a digital image or a photograph) of a portion of the subject's body and/or a topological plot mapping contours of a portion of subject's body. Since the image is “representative” of the external body condition, the image could be either an actual image showing the condition or an image including symbolizations of the condition, for example.

Although mechanisms for receiving the representation are not necessarily a part of the invention in its broadest sense, the representation may be received (and/or initially obtained) by using a web camera, film camera, analog camera, digital camera, scanner, ultra-sound imaging device or any other mechanism for acquiring a representation of the subject's external body. The representation may be received electronically or physically. Examples of electronic means for receiving the representation include reception over a network, reception on a storage medium, facsimile reception, or reception in physical form. Examples of networks that may be used to receive the representation nclude public networks such as the Internet, telephony networks, courier networks (e.g. postal service, United Parcel Service, Federal Express, etc.), private networks, virtual private networks, local area networks, metropolitan area networks, wide area networks, ad hoc networks, or any other mechanism for permitting communication between remote sites, regardless of whether the connection is wired or wireless.

In one example, the receiving of the representation could include receiving a data storage device storing the representation. Examples of data storage devices that may be received include magnetic storage device such as floppy disks, optical storage devices, such as compact discs and digital video discs, organic storage devices, random access memory, printed media, or any other medium for storing information. In another example, hard copies of images may be received and then scanned to convert them into an image in digital form.

In one embodiment, the method may include enabling the subject to receive (e.g., transmitting to the subject) instructions relating to obtaining of the representation. Instructions relating to obtaining of the representation may include instructions relating to capturing of an image with an image capture device chosen from a web cam, digital camera, and a scanner. Such instructions may be provided via text displayed on a display attached to a user access device or via other techniques, such as audio or video techniques. The instructions might also be provided in the form of printed material appearing in hard copy form. After the subject captures the image with the image capture device, the captured image may be transmitted so that the image may be received electronically.

As mentioned above, and shown in FIG. 1, the method may further include maintaining, in a database, information on how use of at least one beauty product affects evolution of the external body condition (step S.20). As used herein, a “database” could include a single data storage structure or a plurality of data storage structures. The database could include a single table or several tables which may be a part of a relational database. In a network environment, the database could be maintained on the user side and/or on the host site. In one example, where the representation is received via a network, the database may be maintained at a location remote from where the subject is located.

The information stored in the database may include information, such as coefficients and/or other mathematical variables, that might be used in order to generate a prognosis for particular conditions and/or beauty products, for example. Information contained in databases may include, but is not limited to, graphical generation data, chart generation data, image morphing information, and/or image libraries representing evolution of an external body condition over time. The information contained in the database could be data used in computer graphics techniques, such as those used when modeling a face by using B-spline patches. Alternatively (or additionally), the information in the database may include a plurality of images representing evolution of one or more external body conditions.

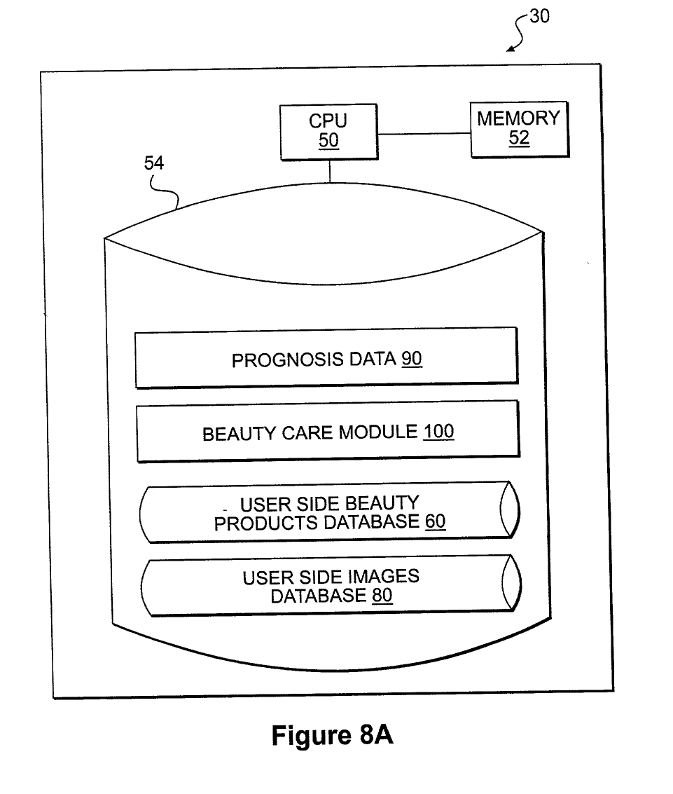

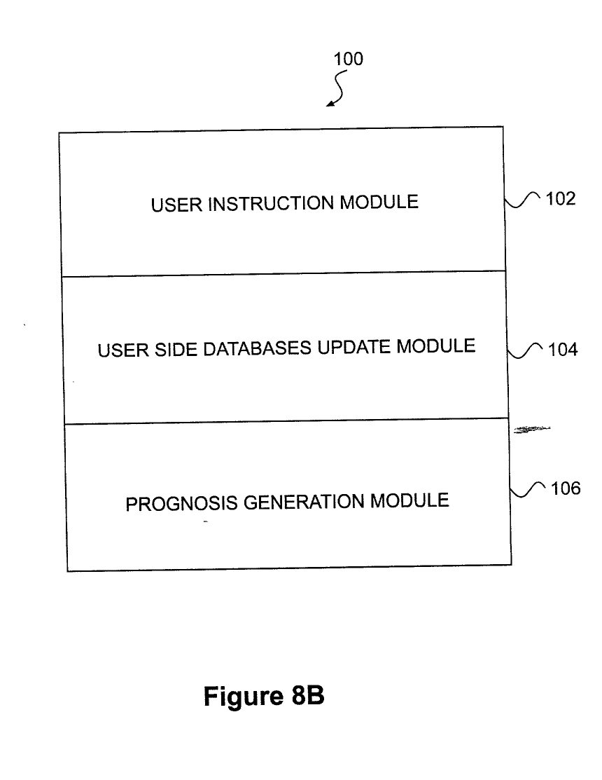

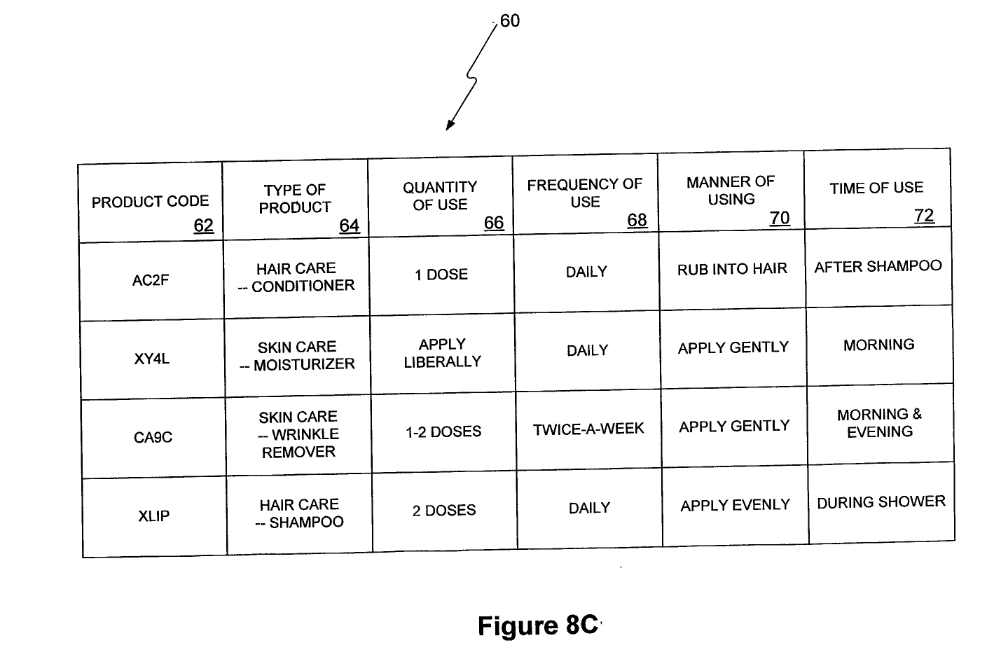

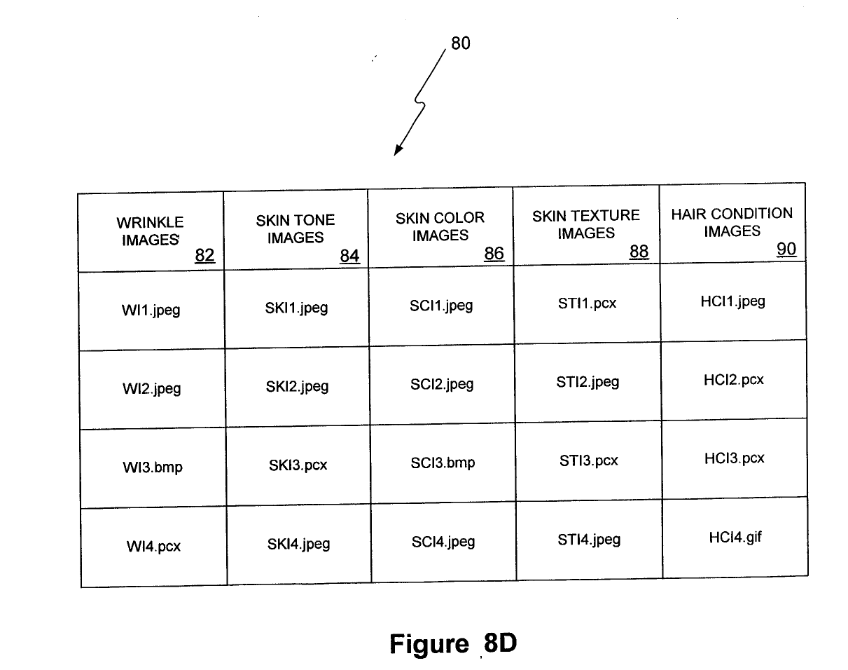

For example, FIG. 8D shows a schematic illustration of a database containing a plurality of differing images of wrinkles 82, skin tone images 84, skin color images 86, skin texture images 88, and hair condition images 90. Wrinkle images 82, such as WI1.jpeg and other such images may be used to depict various degrees of wrinkles in external body locations, such as those on the face. Wrinkle images 82 may also include images that illustrate evolution of wrinkles over time when a beauty product, such as a wrinkle remover, is used. Similarly, skin tone images 84, skin color images 86, skin texture images 88, and hair condition images 90 may include images illustrating varying conditions over times when particular beauty products are used.

Graphics techniques may be used in combination with the database information. For example, attributes, such as texture and/or color, may be stored in a portion of the database for each image and these parameters may be used to construct images at a subsequent time.

The information maintained in the database could relate to a variety of differing beauty products. As used herein, “beauty products” include substances capable of being applied to an external body portion to address (e.g., care and/or treat) one or more external body conditions and/or one or more causes of external body conditions. The beauty products could be chosen from hair products, skin products, and nail products. Examples of skin products include moisturizers, wrinkle removers, exfoliates, toners, and cleansers. Examples of hair products include conditioners and shampoos. Examples of nail products include cuticle creams, oils, and anti-fungal substances.

The information in the database could be the result of information collected through research involving a number of differing subjects, and at least some of those subject may have at least slightly differing conditions. This information may be updated to include further products and/or further information about how the products impact evolution of external body conditions. In one example, where the method is performed at least partially by a computer implementing a neural network, the database could be updated when the neural network acquires further information.

FIG. 8C shows an schematic illustration of a database including a plurality of subsets of information for differing products. Each product may be identified with a product code 62. For each product, the database may further include information about the type of product 64, quantity of use 66, frequency of use 68, manner of using the product 70, and time of use 72. The information about the type of product 64 may include an indication of whether the product is a hair product, skin product, and/or nail product, and may further include information about the category of the product, such as whether the product is a conditioner, moisturizer, wrinkle remover, and/or shampoo, for example.

Information about quantity of use 66 may refer to the quantity that a subject might use for the external body portion, such as the recommended amount of product for each use. Information about frequency of use 68 may refer to the number of times during a particular period that a subject might use a particular beauty product or a combination of beauty products. Information about the manner of using the product 70 may refer to the way in which a subject might apply a particular beauty product and/or the way in which a beauty technique might be performed. Information about time of use 72 may refer to when a subject might use a particular beauty product, for example, after shampooing, during shower, in the morning, and/or in the evening.

The databases schematically shown in FIGS. 8D and 8C could be coupled with one another to form a single database. It should be understood that these illustrations are merely exemplary and that there are many alternative databases that might be possible. For example, the database could have more rows and/or columns of information or fewer rows and/or columns than those shown in FIGS. 8D and 8C. In another example, the schematic illustration of FIG. 8C could include additional information relating to the length of time while the product is used, the length of time between each product use, an applicator device used to apply the product, and/or the manner of using the applicator device.

As briefly mentioned above, the method shown in the flowchart of FIG. 1 further includes generating at least one prognosis reflecting predicted changes in the external body condition after use of at least one beauty product (S.30). This prognosis generation may be based on both the received representation and the information in the database about how use of one or more beauty products affects evolution of the external body condition.

Similar to the representation, the prognosis may be visually perceptible or electronic data capable of being used to construct something visually perceptible. For example, the prognosis may be image data capable of being used to construct a visual image on a display, a holographic image, or any other type of visual means showing one or more predicted changes. In another example, the prognosis could be information capable of being constructed in the form of a viewable graph and/or chart. In a further example, the prognosis could be a morphed version of a representative image, wherein the morphed image is constructed using image morphing information stored in the database.

In one example where the prognosis is in the form of a prognosis image, the prognosis could be a two-dimensional prognosis image, a three-dimensional prognosis image, or even a greater dimensional prognosis image if other dimensions, such as time, are involved.

The prognosis could be generated in any known manner. In one example, where the representation includes image data, the prognosis image generation may include constructing a viewable representative image and comparing the constructed image to information in the database. In another example, the prognosis image may be generated by comparing the representative image of the subject with information contained in the database and selecting a portion of the information in the database based on the comparison.

When the prognosis is in the form of a three-dimensional image, the image could be constructed in accordance with any known technique. Further techniques are disclosed in a co-pending U.S. patent application entitled: Simulation of an Aesthetic Feature on a Facial Image [attorney docket No.05725.0979, filed on the same day as the present application] and a co-pending U.S. patent application entitled: Analysis Using Three-Dimensional Representation [attorney docket No. 05725.1010, filed on the same day as the present application], both of which are incorporated herein by reference.

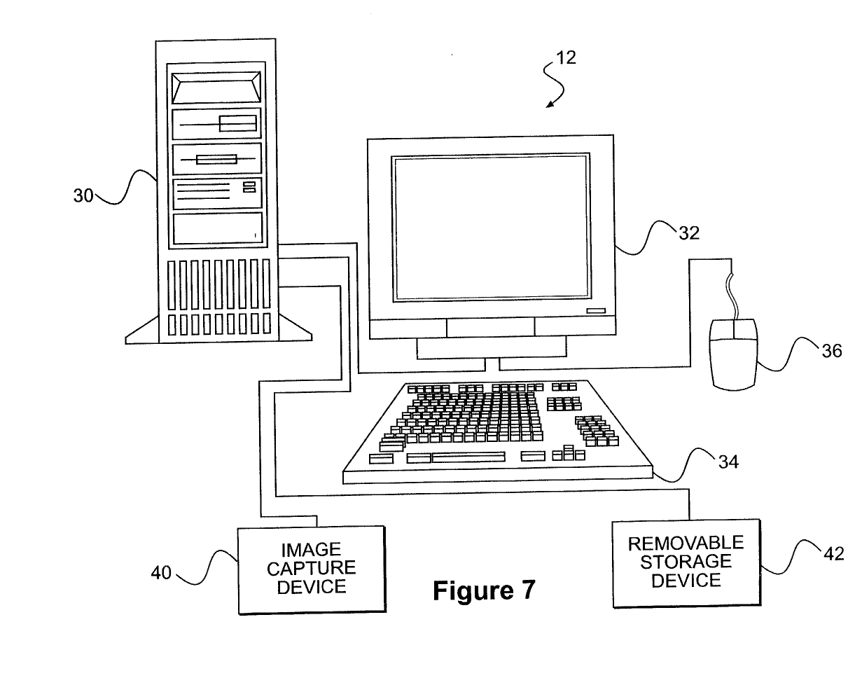

In a further example, the subject could be given the opportunity to select the beauty product from a plurality of differing beauty products (for example, using a user access device, as shown in FIG. 7), and the method could further involve receiving the subject's selection. In such an example, the generation of the prognosis could be based on the product selected by the subject. By way of example, information in the database could be chosen for the prognosis generation based on the subject's product selection.

As shown in the flow chart of FIG. 1, the method further may include outputting the prognosis to enable the subject to receive the prognosis (S.40). The prognosis may be outputted to a display or to any peripheral device, for example, a printer or any other type of peripheral device that permits the subject to view the prognosis. In some examples, the outputting may be performed by transmitting the prognosis. For example, outputting of the prognosis may include transmitting the prognosis via a network from a location located remote from the subject. The outputting of the prognosis may occur through the same means employed in the receipt of the representative image.

FIG. 2 shows an example of one possible form in which output prognosis images could be ultimately displayed to a subject. In this example, a representative image 354 is displayed along with a plurality of prognosis images 356, 358, 360, and 362 showing predicted evolution of an external body condition over time 352. The representative image 354 is the subject's “current” facial image containing a number of wrinkles in the region of the eyes. The prognosis images 356, 358, 360, and 362 are modifications of the subject's facial image showing predicted reductions in the number and intensity of wrinkles over time due of the use of a wrinkle remover product. In this example, if the time 256 is in terms of months, the prognosis image 362 shows substantially complete removal of wrinkles from the subject's face after twelve months of use of the wrinkle removing product, while the intermediate images 356, 358, and 360 show a gradual reduction of wrinkles during the time period. The prognosis images 356, 358, 360, and 362 could be generated using conventional image morphing techniques (e.g., wire mesh morphing) to morph the representative image 354 with image morphing information in the database.

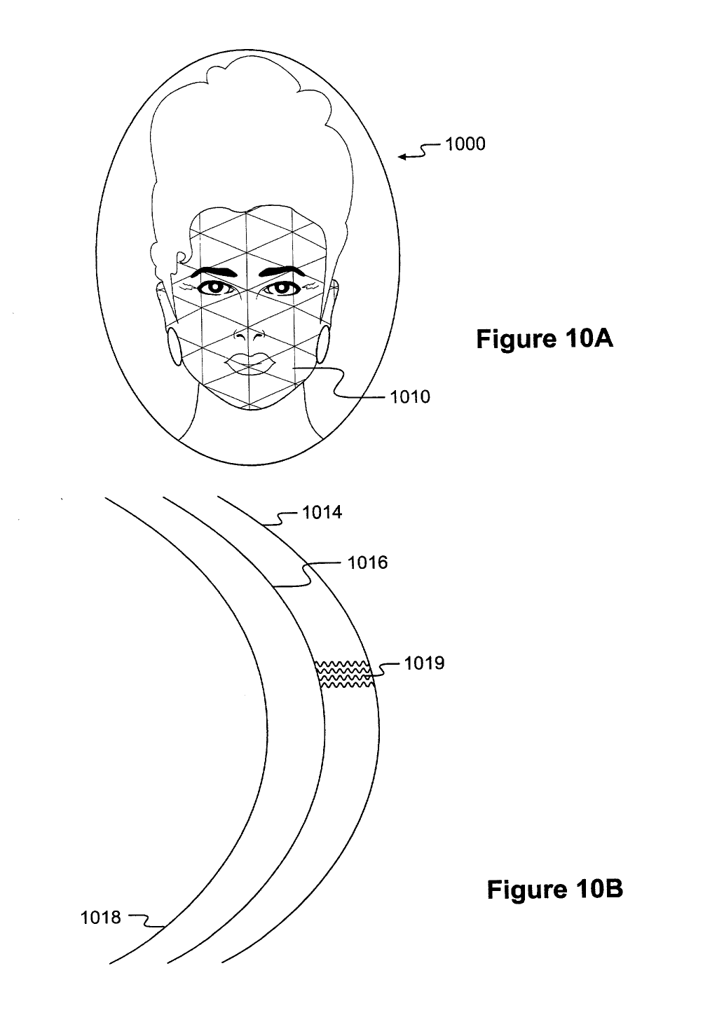

Rather than presenting the representative image 354 and prognosis images 235, 358, 360, and 362 simultaneously, as shown in FIG. 10, each of the images could be displayed to the subject in a one by one format resembling a slide show and/or in a format resembling a movie where the displayed image appears to change while it is displayed.

FIG. 3 shows another example of a possible form in which an output prognosis 306 may be displayed to the subject. In this example, the prognosis 306 is in the form of a graph showing the predicted change of the external body condition over a period of time 302 of using a particular beauty product. The graph 306 represents predicted improvement of skin tone 304 during a period of time 302 in which a skin toning product is used. When the time 302 is in terms of months, the graph 306 could provide the subject with a visual representation enabling the subject to visualize a predicted skin tone improvement of ΔX at 9 months, for example.

As shown in FIG. 3, the displayed information could further include a selectable portion 314 enabling the user to change the period of time 302 from months to years or vice versa, for example. The selectable portion 314 could be a selectable image displayed on a display device.

In addition to outputting the prognosis (S.40), the method may also involve outputting of additional information. For example, the additional output information may include product information enabling the subject to be informed about a beauty product corresponding to the database information used to generate the prognosis. The additional information may also include information relating to beauty product usage assumed to obtain the prognosis. For example, such information may include at least some database information like that shown in FIG. 8C.

In one exemplary embodiment, where the prognosis is in the form of an image, the prognosis image may be rendered on a three-dimensional mesh image. Such rendering may be accomplished using known computer graphics techniques. Also, a mathematical model corresponding to a three-dimensional image resulting from the rendering of the prognosis image on the three-dimensional mesh image may be generated. This may be accomplished using known mathematical technique, such as Fourier transforms, Fast Fourier transforms, or other mathematical techniques. Further, generating the prognosis image may include modifying at least one parameter associated with the mathematical model.

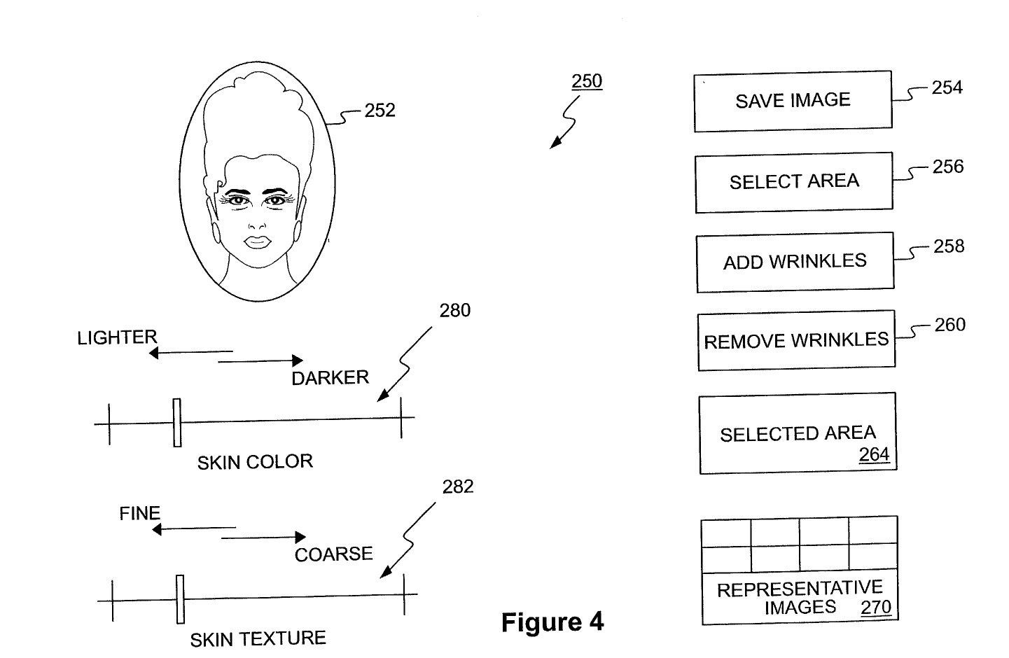

The representative image may be modified by the subject, using for example, an “edit image” user interface 250 depicted in FIG. 4 and explained in more detail below. The user interface could enable the subject to add or remove wrinkles, for example. Addition or removal of wrinkles may modify at least one parameter associated with a mathematical model corresponding to the resulting three-dimensional image.

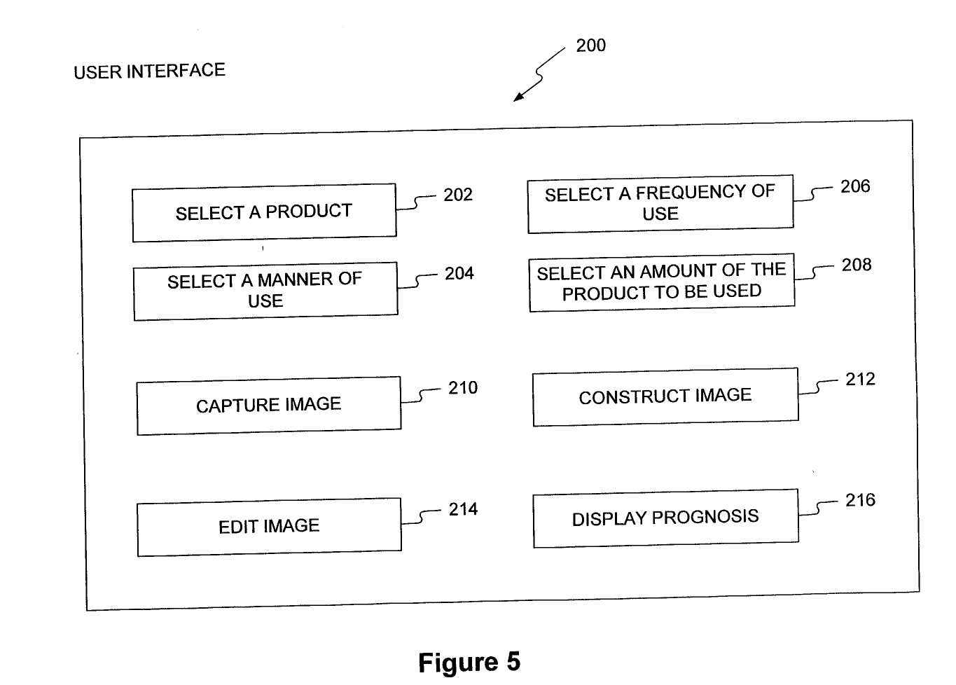

FIG. 5 shows an example of a “menu” interface 200 including several selectable buttons, icons, and/or text commands that may be used to access the functionality associated with a user access device capable of being used by the subject. In one exemplary embodiment user interface 200 may include the following buttons: select a product 202, select a manner of use 204, select a frequency of use 206, select an amount of the product to be used 208, capture image 210, construct image 212, edit image 214, and display of prognosis 216. Using the select a product button 202, the subject may select a beauty product for which the user desires a prognosis. Using the select a manner of use button 204, the subject may select a manner of using the product, such as whether the product should be applied gently or perhaps vigorously. Using the select an amount of product to be used button 208, the subject may select the product amount, for example, one dose or two doses. Activating the capture image button 210 may enable the subject to use an image capture device, such a web cam, to capture a representative image of the subject. A user may also construct a representation using the construct representation button 212. For example, construction of an image by a subject is discussed in above-mentioned co-pending U.S. patent application entitled “Simulation of An Aesthetic Feature on a Facial Image,” attorney docket No. 05725.0979, filed on the same date as the present application. When the image is captured, the user may edit the captured image by selecting the edit image button 214.

FIG. 4 depicts a schematic diagram of an exemplary “edit image” user interface 250 that the user may be presented with once the user selects the edit image button 214 of FIG. 5. Using the “edit image” user interface 250, a subject may edit the representative image to more accurately portray what the subject perceives as being the subject's external body condition. User interface 250 may include a visual representation 252 of the representative image, a control to save the edited image, e.g., save image 254 button. User interface 250 may also include other interface controls, such as select area 256, add wrinkles 258, remove wrinkles 260, selected area 264, representative images 270, a slide bar 280 that may be used to change the skin color, and another slide bar 282 that may be used to change the skin texture, for example, from a finer texture to a coarser texture.

Using the select area 256 control, a user may select a portion of the representative image 252 that the user wants to modify. Then, using the add wrinkles and remove wrinkles controls 258 and 260, the user may add and/or remove wrinkles to/from the selected portion. Alternatively (or additionally), a user may use graphic tools, such as a brush or an eraser icon, that conventional graphics applications provide to modify an image.

Use of the selected area control 264 may enable display of the selected portion, for example, in a zoomed manner. The representative images 270 could include a plurality of differing images that may be selected to modify the representative image 252 with features of those images. For example, the representative images 270 may include a plurality of differing skin tones and selection of one of those skin tones could edit the representative image to include the selected skin tone. Although not shown in FIG. 4, there are many differing types of alternative controls that could be presented to enable the subject to edit the representative image 252.

In another example, the method illustrated in the flow chart of FIG. 1 could involve receipt of a subject's response to one or more queries and use of that response in generating the prognosis. Such queries may relate to personal information about the subject, such as the subject's age, the subject's health, the subject's lifestyle, etc. For example, the subject's response to the queries could be used in selecting the database information used to generate the prognosis.

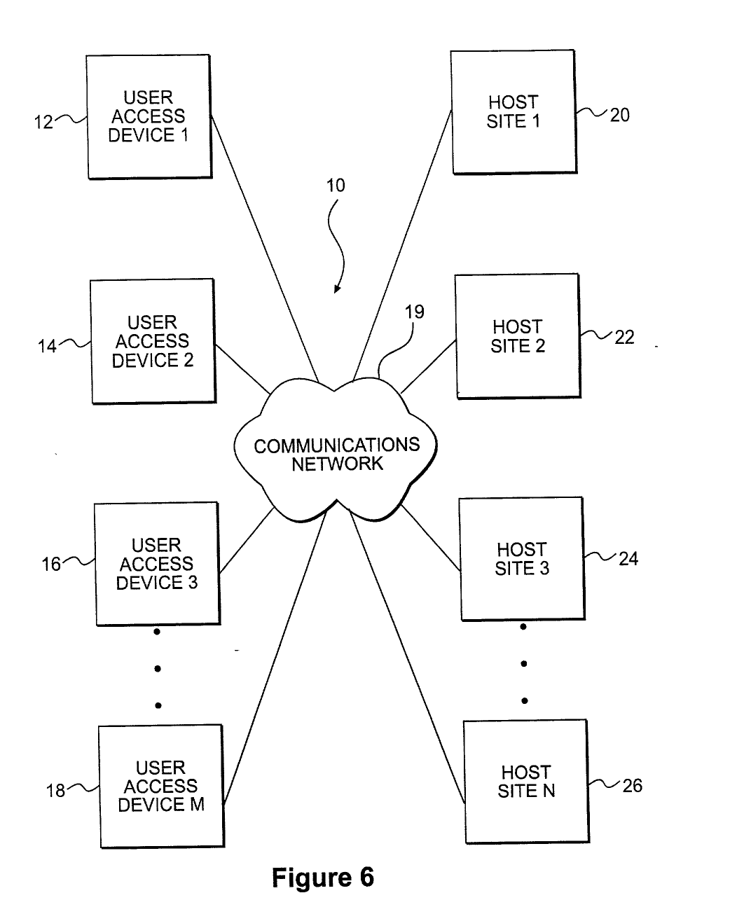

By way of a non-limiting example, FIG. 6 illustrates a system environment 10 in which at least some features and principles of the present invention may be implemented. As illustrated in the schematic diagram of FIG. 6, system environment 10 includes user access devices (1−M) 12-18 connected via a communications network 19 to host sites (1−N) 20-26. Using at least one of the user access devices 12-18, a user such as a consumer of a beauty product may connect to at least one of the host sites 20-26 through the communications network 19. A user access device consistent with the present invention may be based on any computing platform. Such user access devices include, but are not limited to, personal computers, internet access terminals, such as thin client devices, handheld devices, such as palm pilots, or any other device with a computing module. A user access device may be located in various places, including homes, stores, malls, airports, train stations, bus stations or any other location, such as locations where a user may connect to communications network 19 using a wire or a wireless connection.

A host site device consistent with the present invention may be a computing server or an information processing engine located anywhere. The host site device may be connected to at least one user access device via communications network 19.

Communications network 19 may comprise, alone or in any suitable combination, a telephony-based network (such as a PBX or POTS), a local area network (LAN), a wide area network (WAN), a dedicated Intranet, and/or the Internet. Furthermore, any suitable combination of wired and/or wireless components and systems may be incorporated into communications network 19.

Although FIG. 6 depicts each user access device connected to communications network 19, it need not be so. Instead of exchanging information with the host site using the communications network, a user may simply exchange the information using a removable storage device (item 42 shown in FIG. 7), such as an Iomega zip drive, or a memory stick device. Similarly, the host site need not be connected to communications network 19, but instead may exchange information with a user access device through a removable storage device. Information, such as representations, prognoses, prognosis information, or user input could be supplied, conveyed, transmitted, and received in any known manner including any form of electronic communication, such as an e-mail message, a website on an electronic network, and/or a facsimile transmission. The information could also be sent via any non-electronic communication means, such as conventional postal delivery.

Referring now to FIG. 7, a user access device may include a computing module 30, a display 32, a keyboard 34, a pointing device 36, such as a mouse, an image capture device 40 and a removable data storage device 42. Even though FIG. 7 illustrates an exemplary user access device based on a personal computer platform, the user access device may be implemented using a hand-held device platform or any other type of computing platform. Thus, for example, the various components depicted in FIG. 7 may all be combined in any type of combination to provide a user access device. Specifically, for example, using a hand-held device platform one may provide the display, the keyboard, the pointing device, the image capture device, and the movable data storage device on a combined platform, such as a palm pilot hand-held computing device. In other words, the aforementioned description is not meant to be exhaustive; it being understood that the term “user access device,” as used herein, may relate to any type of device that may be used by a subject to access (e.g., receive) and/or supply (e.g., transmit) at least some information associated with the method described above.

FIG. 8A shows an exemplary embodiment of a computing module 30 of the user access device 12 of FIG. 7. Computing module 30 may include at least one CPU 50, at least one memory 52, and at least one storage device 54. Storage device 54, in an exemplary embodiment, may further include a user side beauty products database 60 (e.g., the database shown in FIG. 8C), user side images database 80 (e.g., the database shown in FIG. 8D), prognosis data 90, and beauty care module 100.

Referring now to FIG. 8B, beauty care module 100 of FIG. 8A may include user instruction module 102, user side databases update module 104, and prognosis generation module 106. It may also include a driver for outputting the prognosis (not shown). Alternatively, beauty care module 100 may interface with an operating system associated with the user access device to enable outputting of the prognosis. User instruction module 102 may be used to provide instructions to a user of the user access device. Thus, for example, the user instruction module 102 alone or in combination with other components or programs associated with the computing module may provide a user interface to a user. User side databases update module 104 may be used to intermittently connect a user access device to a host site such that the user side databases may be updated by downloading information stored at the host site. Such updates of the database located at the user site may be initiated by the user or they may occur periodically based on an algorithm. A user may change the frequency of such updates. Prognosis generation module 106 may process a captured image obtained through an image capture device and it may also process the captured image to generate a prognosis image which may then be, for example, displayed on any type of display associated with a user access device.

Although information used by the system may be generally described as being stored in a storage device, one skilled in the art will appreciate that information may be stored on or read from various computer-readable media, such as secondary storage devices, like hard disks, floppy disks, and CD-ROM; a carrier wave received from a network like the Internet; or other forms of ROM or RAM. In one example, instructions for the beauty care module 100 may be downloaded from a remote location. Additionally, it should be noted that the components of the user access device depicted in FIGS. 7, 8A, and 8B are merely exemplary. For example, the user access device may contain additional, fewer, and/or differing components.

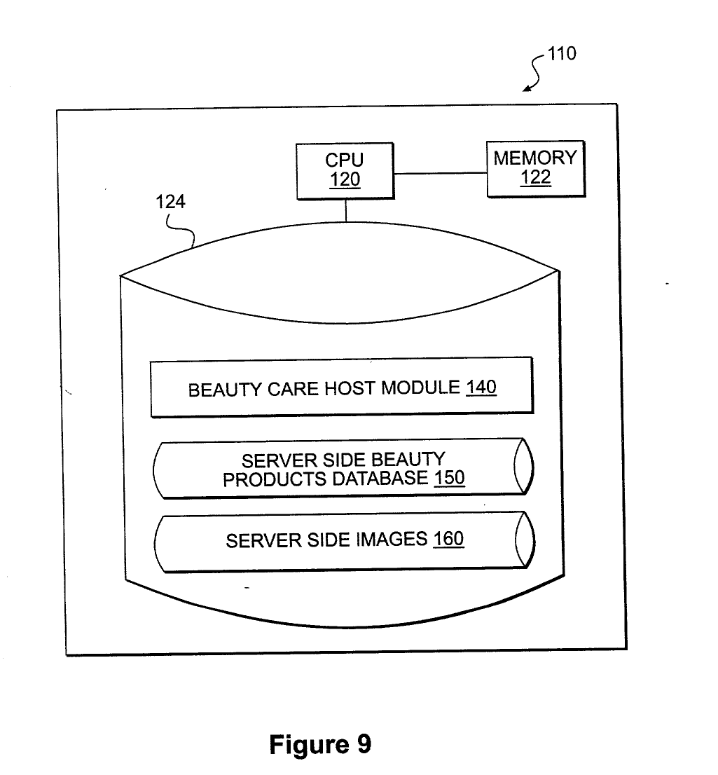

FIG. 9 illustrates an exemplary host site 110. Host site 110 may include at least one CPU 120, at least one memory 122, and at least one storage device 124. Storage device 124, in an exemplary embodiment, may further include a beauty care host module 140, a server side beauty products database 150, and a server side images database 160. Beauty care host module 140 when executed by CPU 120 may interface with the user side beauty care module 100. Although not shown, one skilled in the art will appreciate that beauty care host module 140 may be an application program that may interface with other components such as a web server software and other Internet-related application and/or networking software to enable communication with communications network 19.

Server side beauty products database 150 could be configured to include information such as that shown in FIG. 8C. An operator of the host site may update the server side beauty products database 150 periodically.

Server side images database 160 could be configured to include information such as that shown in FIG. 8D. An operator of the host site may update the images stored in the server side images database 160 periodically.

Computer graphics techniques may be used to depict a representative image of the subject's external body condition. Such techniques may also be used to model the evolution of the external body condition over time. For example, a three dimensional or a two dimensional image of a human face may be created which may be defined by its edges or points. Next, those points may be linked together by lines to create a wire-frame rendering of the object representing the human face. In an exemplary embodiment, an MPEG-4 facial mesh characterized by Facial Definition Parameters (FDPS) may be used. Next, a two-dimensional image representing the external condition of the subject may be applied at the surface of the wire-frame. In some cases objects may be lit by a light source and may be shaded. Surfaces may be represented as polygons, or as B-spline patches, or by any other computer graphics technique. Any graphics application, such as OpenGL, Renderman, or VRML may be used for modeling an external body condition on a human anatomy.

FIG. 10A depicts a model of a human face with B-spline patches representing muscle patches on the representation of the human face 1000. B-spline patch 1010 represents a muscle patch on the exemplary human face 1000. As part of representing facial muscles as B-spline patches, the nature and direction of muscle fibers may be taken into account. Connecting tissue joining the skin to the facial muscle may be simulated using a layer of springs between the skin and the muscle layer.

FIG. 10B depicts a facial simulation (e.g., a facial model) corresponding to a human face. Specifically, a human face may be modeled by noting that it is a layered structure composed of a skull, a muscle layer, an outer skin layer, and connecting tissue between the muscle layer and the outer skin layer. As shown in FIG. 10B, outer skin layer 1014 is connected with muscle layer 1016 through connective tissue which may be modeled by springs 1019 and muscle is further connected to bone 1018. Such a model is discussed in “A Plastic-Visco-Elastic Model for Wrinkles in Facial Animation and Skin Aging,” by Wu et al., which is incorporated by reference in its entirety herein. Using this facial model in one exemplary embodiment, deformations associated with movements of face may be represented. Not only the elastic aspect of facial movement but also the plasticity of skin, which may develop with aging resulting in wrinkles, may also be incorporated as part of this facial model.

As mentioned above, facial muscle structure may be represented by using B-spline patches. In general, the facial muscles are of two types: linear muscles and sphincter muscles. A linear muscle contracts toward an attachment on the bone such as the frontalis major muscle that raises the eyebrows. A sphincter muscle on the other hand, contacts around an imaginary central point such as the orbicularis oris muscle that draws them out together. In one exemplary embodiment, open B-spline patches may be used to simulate the linear muscles while closed B-spline may be used to simulate the sphincter muscles.

Using a modified version of the afore-mentioned model, in one exemplary embodiment, external body conditions, such as wrinkles may be simulated. An addition of a wrinkle may be used as an input to an existing mathematical model of the facial image, and the facial image may be modified accordingly. For example, a plasticity weighting factor associated with the part of the facial image where the wrinkle is to be added may be changed to cause simulation of the addition of the wrinkle. Once a user selects a beauty product, the mathematical model associated with the image may be modified to take into account the effect of the selected beauty product.

Other models and/or mathematical techniques may be used to simulate the affects of beauty products and optionally also the affects of aging. For example, rather than physically-based models, such as the one discussed above, geometric models may be used to simulate an external body condition.

This application may discuss beauty products in connection with use by women. However, it is to be understood that such discussions are for exemplary purposes only. It is to be understood that the invention is equally applicable to all genders, and is not necessarily limited to the beauty industry. It is also to be understood that any functional aspect of the invention can be implemented via any location in the system or network, and data software may be resident at any location either in a network, at a stand-alone site, or on media in the custody and control of a user or subject.

It is to be further understood that the physical mechanisms (e.g. hardware, software, networks, systems) for implementing the methods of the invention are many. Networks, hardware and systems can be configured in a host of ways with software and hardware functionality residing at many alternative locations. In addition, systems other than the exemplary systems disclosed might be used to implement the invention. Therefore, it is to be understood that the methods of the invention are not limited to any particular structure.

Further, methods or portions thereof can be implemented in either an electronic environment, a physical environment, or combinations thereof. Thus, for example, although one or more portions of a method may occur in an electronic environment, a “purchase” portion of the method may occur in a brick and mortar store, or vice versa.

### Cross-Reference to Concurrently Filed Applications and Global Definitions

This application claims priority on and incorporates by reference the following U.S. Provisional applications: Artificial Intelligence For Use In Cosmetic And Non-Cosmetic Environments, Application No. 60/325,561 (provisional filed Oct. 1, 2001); and Methods And Systems For Cosmetic And Non-Cosmetic Product Selection, Application No. 60/325,559 (provisional filed Oct. 1, 2001).

The following concurrently filed U.S. patent applications are also incorporated herein by reference: Body Image Enhancement, Attorney Docket No. 05725.0972; Methods And Systems For Predicting And/Or Tracking Changes In External Body Conditions, Attorney Docket No. 05725.0973; Historical Beauty Record, Attorney Docket No. 05725.0975; Identification And Presentation Of Analogous Beauty Case Histories, Attorney Docket No. 05725.0976; Interactive Beauty Analysis, Attorney Docket No. 05725.0977; Feature Extraction In Beauty Analysis, Attorney Docket No. 05725.0978; Simulation Of An Aesthetic Feature On A Facial Image, Attorney Docket No. 05725.0979; Beauty Advisory System And Method, Attorney Docket No. 05725.0980; Virtual Beauty Consultant, Attorney Docket No. 05725.0981; Calibrating Image Capturing, Attorney Docket No. 05725.0982; Use Of Artificial Intelligence In Providing Beauty Advice, Attorney Docket No. 0572.0983; Shop-In-Shop Website Construction, Attorney Docket No. 05725.0984; Early Detection Of Beauty Treatment Progress, Attorney Docket No. 05725.0985; Cosmetic Affinity Indexing, Attorney Docket No. 05725.0986; Systems And Methods For Providing Beauty Guidance, Attorney Docket No. 05725.0987; Methods And Systems Involving Simulated Application Of Beauty Products, Attorney Docket No.05725.1008; Customized Beauty Tracking Kit, Attorney Docket No. 05725.1009; Analysis Using Three-Dimensional Facial Image Attorney Docket No. 05725.1010; Body Image Templates With Pre-Applied Beauty Products, Attorney Docket No. 05725.1011; and Image Capture Method, Attorney Docket No. 05725.1012.

To the extent not inconsistent with the invention defined herein, definitions and terminology usage in the above-mentioned concurrently filed applications, the above-mentioned priority applications, and the following global definitions are to be considered in interpreting the language of this patent and the claims herein. Where multiple definitions are provided, they should be considered as a single cumulative definition.

The term “image” may include one or more of two-dimensional and three-dimensional representations. In certain examples consistent with the invention, a plurality of images from different perspectives may be used to construct a three-dimensional image. In a broader sense, only a single image may be used. Depending on the embodiment, the term “image” may include either a visually perceptible image or electronic image data that may be either used to construct a visually perceptible image or to derive information about the subject. The image may be a body image corresponding to an anatomical portion of the subject, and may represent, for example, the subject's entire face, or a portion of the subject's face. The image may be a detailed picture (e.g., a digital image or a photograph) of a portion of the subject's body and/or a topological plot mapping contours of a portion of subject's body. If the image is representative of an external body condition, the image could be either an actual image showing the condition or an image including symbolizations of the condition, for example. The image may be an actual or a simulated image. Simulated images may include wholly or partially generated computer images, images based on existing images, and images based on stored features of a subject.

The term “image capture device”, similar terms, and terms representing structures with similar functions may include one or more of a digital camera, webcam, film camera, analog camera, digital video camera, scanner, facsimile machine, copy machine, infrared imager, ultra-sound imaging device, or any other mechanism for acquiring an image of a subject's external body condition, an image of the subject's countenance, an/or an image of the subject's skin. An ultrasonic device might provide skin thickness information, or it might create a map on an area of the external location. Thus, the term “image” as used herein may be broader than a picture. Combinations of image capture devices may be used. For example, an image captured on photographic paper using a film camera might then be scanned on a flat bed scanner to create another image.

The term “capturing (an image)”, or any form thereof, refers to the use of an image capture device to acquire an image. “Capturing” may refer to the direct act of using the image capture device to acquire the image. It may also include indirect acts to promote acquisition. To this end, “capturing” may include the indirect acts of providing access to hardware, or to at least one of a client-based algorithm and a server-based algorithm for causing the image capture device to capture an image. This may be accomplished by providing a user with software to aid in the image capture process, or providing the user with access to a network location at which the software resides. Also consistent with certain embodiments of the invention, capturing may include at least one of receiving an instruction from the subject to capture an image, indicating to the subject before the image is captured, and indicating to the subject when the image is captured.

The term “image processing technique” or similar terms, may include a software program, computer, application specific integrated circuit, electronic device and/or a processor designed to identify in an image one or more characteristics, such as a skin condition. Such techniques may involve binarization, image partitioning, Fourier transforms, fast Fourier transforms (FFTs), and/or discrete cosine transforms may be performed on all or part of the image, resulting in coefficients. Based on the coefficients, conditions may be located, as known in the art. Artificial intelligence, such as fuzzy logic, neural networks, genetic programming and decision tree programming, may also be used to identify conditions. Alternatively, one or more digital filters may be passed through the image for locating specific conditions. These examples are provided for illustrative purposes with the understanding that any image processing technique may be used.

The term “network interface” or similar terms, refer to any mechanism for aiding communications between various nodes or locations in a network. A network interface may include, for example a bus, a modem, or any other input/output structure. A network interface may permit a connection to any network capable of being connected to an input and/or output module located within at least one or more of the following exemplary networks: an Ethernet network, an Internet Protocol network, a telephone network, a radio network, a cellular network, or any mechanism for permitting communication between two or more modes or remote locations. In some invention embodiments, a network interface might also included a user interface.

The term “user interface” may include at least one component such as a keyboard, key pad, mouse, track ball, telephone, scanner, microphone, touch screen, web cam, interactive voice response system (IVR), voice recognition system or any other suitable input mechanism for conveying information. A user interface may also include an input port connected by a wired, optical, or wireless connection for electromagnetic transmissions. In some embodiments, a user interface may include connections to other computer systems to receive the input commands and data therefrom. User interface may further include a data reading device such as a disk drive for receiving input data from and writing data to storage media such as magnetic and optical disks.

As used herein terms such as “external body condition”, “skin condition”, and “actual condition” refer to conditions of at least one of the skin, teeth, hair, eyebrows, eyelashes, body hair, facial hair, fingernails, and/or toenails, or any other externality. Examples of skin conditions may include elasticity, dryness, cellulitis, sweating, aging, wrinkles, melanoma, exfoliation, desquamation, homogeneity of color, creases, liver spots, clarity, lines, micro-circulation, shininess, softness, smoothness, tone, texture, matitty, hydration, sag, suppleness, stress, springiness, firmness, sebum production, cleanliness, translucency, luminosity, irritation, redness, vasocolation, vasomotion, vasodilation, vasoconstriction, pigmentation, freckles, blemishes, oiliness, pore distribution, pore size, moles, birthmarks, acne, blackheads, whiteheads, pockmarks, warts, pustules, boils, blisters, marks, smudges, specks, psoriasis and other characteristics associated with the subject's skin. Examples of hair conditions may include keratin plug, length, dryness, oiliness, dandruff, pigmentation, thickness, density, root conditions, split ends, hair loss, hair thinning, scales, staging, cleanliness and other properties related to the subject's hair. Examples of fingernail and toenail conditions may include onychomycosis, split nails, delaminating, psoriasis, brilliancy, lines, spots, coloration, gloss, strength, brittleness, thickness, hangnail, length, disease, and other characteristics related to the subject's nails. Other conditions may include, for example, size and proportion of facial features, teeth discoloration, and any other aesthetic-related or physical, physiological, or biological conditions of the user.

“Enabling”, “facilitating”, and “causing” an action refer to one or more of a direct act of performing the action, and any indirect act of encouraging or being an accessory to the action. Thus, the terms include partnering or cooperating with an entity who performs the action and/or referring commerce to or having commerce referred from an entity who performs the action. Other examples of indirect activity encompassed within the definitions of “enabling”, “facilitating”, and “causing” may include providing a subject with one or more of tools to knowingly aid in performing the action, providing instructions on how to perform the action, providing prompts or cues to perform the action, or expressly encouraging performance of the action. Indirect activity may also include cooperating with an entity who either directly performs the action or who helps another perform the action. Tools may include software, hardware, or access (either directly, through hyperlink, or some other type of cooperation or partnering) to a network location (e.g., web site) providing tools to aid in performing the action. Thus, phrases such as “enabling access” and “enabling display” do not necessary require that the actor actually access or display anything. For example, the actor may perform the enabling function by affiliating with an entity who performs the action, or by providing instructions, tools, or encouragement for another to do the accessing and displaying.

Forms of the word “displaying” and like terms may also include indirect acts such as providing content for transmission over a network to a display device, regardless of whether the display device is in the custody or control of the sender. Any entity in a chain of delivering information for display performs an act of “displaying”, as the term is used herein.

Likewise, the term “providing” includes direct and indirect activities. For example, providing access to a computer program may include at least one of providing access over a network to the computer program, and creating or distributing to the subject a computer program configured to run on the subject's workstation or computer. For example, a first party may direct network traffic to (either through electronic links or through encouragement to visit) a server or web site run by a second party. If the second party maintains a particular piece of software thereon, then it is to be understood that within the meaning of “providing access” as used herein, the first party is said to provide access to the particular software. Or if the first party directs a subject to a second party who in turn ships the particular software to the user, the first party is said to provide the user with access to the particular software. (Of course, in both of the above instances, the second party would also be providing access within the meaning of the phrase as used herein.) “Receiving” may include at least one of acquisition via a network, via verbally communication, via electronic transmission, via telephone transmission, in hard-copy form, or through any other mechanism enabling reception. In addition, “receiving” may occur either directly or indirectly. For example, receipt may occur through a third party acting on another party's behalf, as an agent of another, or in concert with another. Regardless, all such indirect and direct actions are intended to be covered by the term “receiving” as used herein. A received request, for example, may take one of many forms. It may simply be a checked box, clicked button, submitted form or oral affirmation. Or it might be a typed or handwritten textual request. Receiving may occur through an on-line interest form, e-mail, facsimile, telephone, interactive voice response system, or file transfer protocol transmitted electronically over a network at a web site, an internet protocol address, or a network account. A request may be received from a subject for whom information is sought, or an entity acting on the subject's behalf. “Receiving” may involve receipt directly or indirectly through one or more networks and/or storage mediums. Receipt may occur physically such as in hard copy form, via mail delivery or other courier delivery.

Forms of the word “maintain” are used broadly to include gathering, storing, accessing, providing access to, or making something available for access, either directly or indirectly. For example, those who maintain information include entities who provide a link to a site of a third party where the information is stored.

Consistent with the concepts set forth above, all other recited actions such Consistent with the concepts set forth above, all other recited actions such as, for example, obtaining, determining, generating, selecting, applying, simulating, presenting, etc, are inclusive of direct and indirect actions. Thus, for purposes of interpreting the following claims, an entity performs a recited action through either direct or indirect activity. Further examples of indirect activity include sending signals, providing software, providing instructions, cooperating with an entity to have the entity perform the action, outsourcing direct or indirect actions, or serving in any way as an accessory to the specified action.

The term “product” is used to generically refer to tangible merchandise, goods, services and actions performed. A “beauty product,” “beauty care product,” “cosmetic product” or similar terms, refer to products (as defined above) for effecting one or more external body conditions, such as conditions of the skin, hair and nails. Examples of tangible merchandise forms of beauty products include cosmetic goods, such as treatment products, personal cleansing products, and makeup products, in any form (e.g., ointments, creams, gels, sprays, supplement, ingesta, inhalants, lotions, cakes, liquids, and powders.)

Examples of services forms of beauty products include hair styling, hair cutting, hair coloring, hair removal, skin treatment, make-up application, and any other offering for aesthetic enhancement. Examples of other actions performed include massages, facial rubs, deep cleansings, applications of beauty product, exercise, therapy, or any other action effecting the external body condition whether performed by a professional, the subject, or an acquaintance of the subject.

The following is exemplary and non-exhaustive listing of a few beauty products- scrubs, rinses, washes, moisturizers, wrinkle removers, exfoliates, toners, cleansers, conditioners, shampoos, cuticle creams, oils, and anti-fungal substances, anti-aging products, anti-wrinkle products, anti-freckle products, skin conditioners, skin toners, skin coloring agents, tanners, bronzers, skin lighteners, hair coloring, hair cleansing, hair styling, elasticity enhancing products, agents, blushes, mascaras, eyeliners, lip liners, lipsticks, lip glosses, eyebrow liners, eye shadows, nail polishes, foundations, concealers, dental whitening products, cellulite reduction products, hair straighteners and curlers, and weight reduction products. A beauty care treatment regimen may involve the administration of one or more products, as defined above.

The terms “beauty advice”, “beauty guidance”, and similar terms are used interchangeably to refer to the provision of beauty related information to a subject. Advice or guidance includes one or more of beauty product recommendations (e.g., cosmetic product recommendations for products to treat conditions the subject is prompted to evaluate), remedial measures, preventative measures, predictions, prognoses, price and availability information, application and use information, suggestions for complementary products, lifestyle or dietary recommendations, or any other information intended to aid a subject in a course of future conduct, to aid a subject in understanding past occurrences, to reflect information about some future occurrences related to the subject's beauty or to aid a subject in understanding beauty products, as defined above.

The term “network” may include a public network such as the Internet or a telephony network, a private network, a virtual private network, or any other mechanism for enabling communication between two or more nodes or locations. The network may include one or more of wired and wireless connections. Wireless communications may include radio transmission via the airwaves, however, those of ordinary skill in the art will appreciate that various other communication techniques can be used to provide wireless transmission including infrared line of sight, cellular, microwave, satellite, blue-tooth packet radio and spread spectrum radio. Wireless data may include, but is not limited to, paging, text messaging, e-mail, Internet access and other specialized data applications specifically excluding or including voice transmission.

In some instances consistent with the invention, a network may include a courier network (e.g. postal service, United Parcel Service, Federal Express, etc.). Other types of networks that are to be considered within the scope of the invention include local area networks, metropolitan area networks, wide area networks, ad hoc networks, or any mechanism for facilitating communication between two nodes or remote locations.

“Artificial intelligence” (AI) is used herein to broadly describe any computationally intelligent systems that combine knowledge, techniques, and methodologies. An AI engine may be any system configured to apply knowledge and that can adapt itself and learn to do better in changing environments. Thus, the AI engine may employ any one or combination of the following computational techniques: neural network, constraint program, fuzzy logic, classification, conventional artificial intelligence, symbolic manipulation, fuzzy set theory, evolutionary computation, cybernetics, data mining, approximate reasoning, derivative-free optimization, decision trees, or soft computing. Employing any computationally intelligent techniques, the AI engine may learn to adapt to unknown or changing environment for better performance. AI engines may be implemented or provided with a wide variety of components or systems, including one or more of the following: central processing units, co-processors, memories, registers, or other data processing devices and subsystems.

AI engines may be trained based on input such as product information, expert advice, user profile, or data based on sensory perceptions. Using input an AI engine may implement an iterative training process. Training may be based on a wide variety of learning rules or training algorithms. For example, the learning rules may include one or more of the following: back-propagation, real-time recurrent learning, pattern-by-pattern learning, supervised learning, interpolation, weighted sum, reinforced learning, temporal difference learning, unsupervised learning, or recording learning. As a result of the training, AI engine may learn to modify its behavior in response to its environment, and obtain knowledge. Knowledge may represent any information upon which AI engine may determine an appropriate response to new data or situations. Knowledge may represent, for example, relationship information between two or more products. Knowledge may be stored in any form at any convenient location, such as a database.

Since AI engine may learn to modify its behavior, information describing relationships for a universe of all combinations of products may not need to be maintained by the AI engine or any other component of the system.

“Personal information”, “subject specific information”, “user specific information”, “user profile”, “personal characteristics”, “personal attributes”, “profile information”, and like terms (collectively referred to in this section as “personal information”) may broadly encompass any information about the subject or user. Such information may, for example, fall within categories such as physical characteristics, fashion preferences, demographics, nutritional information, cosmetic usage information, medical history information, environmental information, beauty product usage information, lifestyle, and may include information such as name; age; birth date; height; weight; ethnicity; eating habits; vacation patterns; geographic location of the individual's residence, location, or work; work habits; sleep habits; toiletries used; exercise habits; relaxation habits; beauty care habits; smoking and drinking habits; sun exposure habits; use of sunscreen; propensity to tan; number of sunburns and serious sunburns; dietary restrictions; dietary supplements or vitamins used; diagnosed conditions affecting the external body, such as melanoma; an image, such as a picture or a multimedia file of the subject; facial feature characteristics; family history information such as physical characteristics information about relatives of the subject (e.g., premature balding, graying, wrinkles, etc.); external body condition (as defined previously); color preferences, clothing style preferences, travel habits; entertainment preferences; fitness information; adverse reactions to products, compounds, or elements (e.g., sun exposure); body chemistry, use of prior beauty care products and their effectiveness; purchasing, shopping, and browsing habits; hobbies; marital status; whether the subject is a parent; country of residence; region of residence; birth country and region; religious affiliation; political affiliation; whether the subject is an urban dweller suburban dweller or rural area dweller; size of urban area in which the subject lives; whether the subject is retired; annual income, sexual preference, or any other information reflecting habits, preferences, or affiliations of the subject.

Personal information may also include information electronically gleaned by tracking the subject's electronic browsing or purchasing habits, or as the result of cookies maintained on the subject's computer, responses to surveys, or any other mechanism providing information related to the subject. In addition, personal information may be gathered through non-electronic mechanisms such as hard copy surveys, personal interviews, or consumer preference polls.

“Complementary” and “complementary product” refers to one or more of physical, physiological, biologically, and aesthetic compatibility. A product may be complementary with one or more of another product, a group of products, or a subject. In that latter instance, whether a product is considered “complementary” may be a function of personal information of the subject. Thus, for example a product may be complementary if it is unlikely to cause an adverse allergic reaction; if it physically blends well with another product; or if it is aesthetically consistent with the subject or one or more other products. Aesthetic compatibly may refer to the fact that two products are aesthetically appealing (or do not clash) when worn together. The identification of a complementary product may also be based on product characteristics, user preferences, survey data, or expert advice.

As used herein, the words “may” and “may be” are to be interpreted in an open-ended, non-restrictive manner. At minimum, “may” and “may be” are to be interpreted as definitively including structure or acts recited. Further, the word “or” is to be interpreted in the conjunctive and the disjunctive.

While flow charts presented herein illustrate a series of sequential blocks for exemplary purposes, the order of blocks is not critical to the invention in its broadest sense. Further, blocks may be omitted and others added without departing from the spirit of the invention. Also, the invention may include combinations of features described in connection with differing embodiments.

Although a focus of the disclosure may be on server-side methods, it is nevertheless to be understood that the invention includes corresponding client-side methods, software, articles of manufacture, and computer readable media, and that computer readable media can be used to store instructions for some or all of the methods described herein. Further, it is to be understood that disclosed structures define means for implementing the functionality described herein, and that the invention includes such means for performing the disclosed functions.

In the foregoing Description of Exemplary Embodiments, various features are grouped together in a single embodiment for purposes of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Description of the Exemplary Embodiments, with each claim standing on its own as a separate embodiment of the invention.

What is claimed is:

1. A method for enabling determination of a prognosis for an external body condition of a subject, the method comprising:
receiving at least one representation of the subject's external body condition;
maintaining in a database information of how use of at least one beauty product affects evolution of the external body condition;
generating at least one prognosis reflecting predicted changes in the external body condition after use of said at least one beauty product, wherein the generating is based on both the received representation and information contained in the database; and
outputting the prognosis to enable the subject to receive the prognosis.

2. The method of claim 1, wherein the at least one representation defines the external body condition.

3. The method of claim 1, wherein the at least one representation comprises at least one image representative of the external body condition.

4. The method of claim 1, wherein the prognosis comprises a prognosis image.

5. The method of claim 4, wherein the prognosis image comprises a three-dimension prognosis image.

6. The method of claim 1, further comprising enabling the subject to receive instructions relating to obtaining of the representation.

7. The method of claim 6, wherein the instructions comprise instructions relating to capturing of an image with an image capture device chosen from a web cam, a digital camera, and a scanner.

8. The method of claim 1, further comprising constructing an image based on the representation, wherein said generating of the prognosis comprises comparing the constructed image to the database information.

9. The method of claim 3, wherein the wherein the database information comprises image morphing information, and wherein said generating of the prognosis comprises morphing the representative image based on the image morphing information.

10. The method of claim 1, wherein the representation is received via a network, and wherein the subject is located at a location remote from a location where the database is maintained.

11. The method of claim 1, wherein the representation is stored in a data storage device, and wherein said receiving of the representation comprises receiving the data storage device.

12. The method of claim 1, wherein the database information comprises a plurality of subsets of information, wherein at least some of the subsets of information relate to differing beauty products.

13. The method of claim 12, wherein the outputting further comprises outputting product information to enable the subject to be informed about a beauty product relating to the database information used to generate the prognosis.

14. The method of claim 1, wherein the database information comprises a plurality of subsets of information, wherein at least some of the subsets of information relate to differing manners of using a beauty product.

15. The method of claim 14, wherein the outputting further comprises outputting usage information to enable the subject to be informed about the manner of beauty product usage relating to the database information used to generate the prognosis.

16. The method of claim 14, wherein the manners of using differ from one another according to at least one of timing of product usage, the length of time while the product is used, frequency of use of the product, the length of time between each use of the product, the manner in which the product is applied, an applicator device used to apply the product, and the manner of using the applicator device.

17. The method of claim 1, wherein the beauty product is chosen from skin products, hair products, and nail products.

18. The method of claim 1, wherein the beauty product comprises a skin product chosen from moisturizers, wrinkle removers, and exfoliates.

19. The method of claim 1, wherein the beauty product comprises a hair product chosen from a conditioner and a shampoo.

20. The method of claim 1, further comprising outputting product purchase information enabling the subject to purchase the beauty product relating to the database information used to generate the prognosis.

21. The method of claim 1, wherein the generating the prognosis comprises comparing the representation with information in the database and selecting a portion of the information in the database based on the comparing.

22. The method of claim 1, wherein the outputting the prognosis comprises transmitting the prognosis via a network.

23. The method of claim 1, wherein the at least one beauty product is selected from a plurality of differing beauty products, and wherein the method further comprises receiving a selection of the at least one beauty product.

24. The method of claim 23, further comprising enabling the subject to make the selection of the at least one beauty product from the plurality of beauty products.

25. The method of claim 1, further comprising rendering the prognosis on a three-dimensional mesh image.

26. The method of claim 25, further comprising generating a mathematical model corresponding to a three-dimensional image resulting from the rendering of the prognosis on the three-dimensional mesh image.

27. The method of claim 3, further comprising enabling modification of the representative image based on an input by the subject.

28. The method of claim 27, wherein the input by the subject further comprises at least one of the addition and removal of wrinkles from the representative image.

29. The method of claim 28, wherein said at least one of the addition and removal of wrinkles comprises modifying at least one parameter associated with a mathematical model corresponding to the image.

30. The method of claim 26, wherein generating at least one prognosis comprises modifying at least one parameter associated with the mathematical model.

31. A system for enabling determination of prognosis for an external body condition of a subject, the system comprising:
a memory for receiving at least one representation of the subject's external body condition;
a database for storing information on how use of at least one beauty product affects evolution of the external body condition;
a processor for modifying the representation, based on information contained in the database, to generate at least one prognosis reflecting predicted changes in the external body condition after use of said at least one beauty product; and
a driver for outputting the prognosis to enable the subject to receive the prognosis.

32. A system for enabling determination of a prognosis for an external body condition of a subject, the system comprising:
means for receiving at least one representation of the subject's external body condition;
means for maintaining in a database information of how use of at least one beauty product affects evolution of the external body condition;
means for generating at least one prognosis reflecting predicted changes in the external body condition after use of said at least one beauty product, wherein the generating is based on both the representation and information contained in the database; and
means for outputting the prognosis to enable the subject to receive the prognosis.

33. The system of claim 32, further comprising means for enabling the subject to receive instructions relating to obtaining of the representation.

34. The system of claim 33, wherein the instructions comprise instructions relating to capturing of an image with an image capture device chosen from a web cam, a digital camera, and a scanner.

35. The system of claim 32, further comprising means for constructing a representative image, wherein said means for generating the prognosis compares the constructed image to the database information.

36. The system of claim 32, wherein the representation comprises at least one image representative of the external body condition, wherein the database information comprises image morphing information, wherein said means for generating the prognosis comprises means for morphing the representative image based on the image morphing information.

37. The system of claim 32, wherein the means for receiving receives the representation image data via a network.

38. The system of claim 32, wherein the representation is stored in a data storage device, and wherein said means for receiving the representation comprises means for receiving the data storage device.

39. The system of claim 32, wherein the database information comprises a plurality of subsets of information, wherein at least some of the subsets of information relate to differing beauty products.

40. The system of claim 39, wherein the means for outputting further comprises means for outputting product information to enable the subject to be informed about a beauty product relating to the database information used to generate the prognosis.

41. The system of claim 32, wherein the database information comprises a plurality of subsets of information, wherein at least some of the subsets of information relate to differing manners of using a beauty product.

42. The system of claim 41, wherein the means for outputting further comprises means for outputting usage information to enable the subject to be informed about the manner of beauty product usage relating to the database information used to generate the prognosis image.

43. The system of claim 41, wherein the manners of using differ from one another according to at least one of timing of product usage, the length of time while the product is used, the frequency of use of the product, the length of time between each use of the project, the manner in which the product is applied, an applicator device used to apply the product, and the manner of using the applicator device.

44. The system of claim 32, further comprising means for outputting product purchase information enabling the subject to purchase the beauty product relating to the database information used to generate the prognosis.

45. The system of claim 32, wherein the means for generating the prognosis comprises means for comparing the representation with information in the database and selecting a portion of the information in the database based on the comparing.

46. The system of claim 32, wherein the means for outputting the prognosis comprises means for transmitting the prognosis via a network.

47. A system for enabling determination of a prognosis for an external body condition of a subject, the system comprising:
a memory for receiving at least one image representative of the subject's external body condition;
a secondary storage storing a mesh frame representative of at least one part of human anatomy;
a database containing information on how use of at least one beauty product affects evolution of the external body condition;
a processor for rendering the image on the mesh frame and for modifying the image, based on information contained in the database, to generate at least one prognosis image reflecting predicted changes in the external body condition after use of the at least one beauty product; and
a driver for outputting the prognosis image to enable the subject to view the prognosis image.

48. A computer-readable medium containing instructions for a method for enabling determination of a prognosis for an external body condition of a subject, the method comprising:
receiving at least one representation of the subject's external body condition;
maintaining in a database information of how use of at least one beauty product affects evolution of the external body condition;
generating at least one prognosis reflecting predicted changes in the external body condition after use of said at least one beauty product, wherein the generating is based on both the representation and information contained in the database; and
outputting the prognosis to enable the subject to receive the prognosis.
